(19) United States
(12) Patent Application Publication
(10) Pub. No.: US 2019/0169653 A1
(43) Pub. Date: Jun. 6, 2019

(54) METHOD FOR PREPARING GENE KNOCK-IN CELLS

(71) Applicant: National University Corporation Tokyo Medical and Dental University, Tokyo (JP)

(72) Inventors: Tomomi AIDA, Tokyo (JP); Kohichi TANAKA, Tokyo (JP)

(73) Assignee: National University Corporation Tokyo Medical and Dental University, Tokyo (JP)

(21) Appl. No.: 16/324,213

(22) PCT Filed: Aug. 1, 2017
(86) PCT No.: PCT/JP2017/027838

§ 371 Date: Feb. 8, 2019

(30) Foreign Application Priority Data
Aug. 10, 2016 (JP) 2016-157484

(51) Int. Cl. C12N 15/90; A01K 67/027; C12N 9/22

(52) U.S. Cl. CPC C12N 15/907; A01K 67/0278; C12N 2310/20; C12N 2800/80; C12N 9/22

(57) ABSTRACT

The present inventors tried to prepare a knock-in animal using a genome editing system containing a nuclease in the form of protein and a DNA-targeting RNA and using a single-stranded DNA as a donor, and found that it is possible to obtain a cell in which the donor DNA has been knocked in with extremely high efficiency.

### TECHNICAL FIELD

The present invention relates to a method for highly efficiently preparing gene knock-in cells by using a genome editing technique such as a CRISPR-Cas9 system.

### BACKGROUND ART

Gene targeted (knock-out or knock-in) mammals serve as an important tool in analyzing gene functions in vivo, but their production requires complex and effort-taking steps of using embryonic stem cells (ES cells).

Genome editing techniques such as ZFN, TALEN, and CRISPR-Cas9 have been developed so far, which are drawing attention as useful tools for genetic modification.

The CRISPR-Cas9 system, currently the most used of these genome editing techniques, is based on bacterial acquired immune mechanism, and the complex composed of a Cas9 protein being a double-stranded DNA cleaving enzyme, an RNA (crRNA) having a nucleotide sequence complementary to the target DNA region, and an RNA having a nucleotide sequence partially complementary to the crRNA (trans-activating RNA; tracrRNA) specifically recognizes and binds the target DNA region for cleavage.

Use of the system makes it possible to produce a gene targeted mammal without involving ES cells by introducing an RNA encoding a Cas9 protein as well as a crRNA and a tracrRNA (including the case of the form of single-stranded chimeric RNA in which these two RNAs are linked via a linker nucleotide) into a fertilized egg and directly manipulating the genome of the fertilized egg in vivo (in vivo genome modification) (Patent Literature 1 and Non Patent Literature 1). To date, this method has been used a number of times to produce knock-out mice (Patent Literature 2 and Non Patent Literatures 2 to 4) and to produce knock-in mice with single base substitution (Non Patent Literatures 3, 5, and 6).

However, in the production of a knock-in mammal into which a gene of a relatively large size has been inserted using a CRISPR-Cas9 system, there is a problem that the gene knock-in efficiency is very low (Non Patent Literature 7).

In view of the above circumstances, a method was developed for highly efficient knock-in into a non-human mammal using a cloning-free CRISPR-Cas9 system composed of Cas9 protein (in the form of protein, not RNA), crRNA fragment, and tracrRNA fragment and using a relatively large-sized double-stranded DNA as a donor (Patent Literature 3). Mainly, this method makes it possible to prepare a mammal in which the donor DNA has been heterozygously knocked in (see Comparative Example 2 to be described later).

On the other hand, a method has also been developed for highly efficient knock-in into a non-human mammal using a single-stranded DNA as a donor and using a conventional CRISPR-Cas9 system (Non Patent Literature 8). This method is successful in obtaining a mammal in which the donor DNA has been homozygously knocked in.

Moreover, the CRISPR-cpf1 system has recently been developed as a new genome editing technique (Non Patent Literature 9). This system is also a genome editing technique which uses a guide RNA and a nuclease that interacts with it as in the case of the CRISPR-Cas9 systems, and is expected to be used in a manner similar to that of the CRISPR-Cas9 systems.

### CITATION LIST

### Patent Literature

[PTL 1] International Publication No. WO 2014/131833
[PTL 2] International Publication No. WO 2013/188522
[PTL 3] International Publication No. WO 2016/080097

### Non Patent Literature

[NPL 1] Aida, T. et al., Dev. Growth Differ. 56, 34-45, 194 (2014).
[NPL 2] Shen, B. et al., Cell Res. 23 720-3 (2013).
[NPL 3] Wang, H. et al., Cell 153, 910-8 (2013).
[NPL 4] Li, D. et al., Nat. Biotechnol. 31, 681-3 (2013).
[NPL 5] Long, C. et al., Science 345, 1184-8 (2014).
[NPL 6] Wu, Y. et al., Cell Stem Cell 13, 659-62 (2013).
[NPL 7] Yang, H. et al., Cell 154, 1370-9 (2013).
[NPL 8] Miura, H. et al., Sci. Rep. 5, 12799 (2015)
[NPL 9] Zetsche, B. et al., Cell 163 (3), 759-71 (2015)

### SUMMARY OF INVENTION

### Technical Problem

The present invention aims to provide a method which is capable of knocking in a donor DNA to cells with a dramatically higher efficiency than that of conventional methods and is capable of homozygously knocking in a donor DNA.

### Solution to Problem

The present inventors have made earnest studies in order to achieve the object described above and tried to prepare a knock-in animal using a CRISPR-Cas9 system composed of Cas9 protein (in the form of protein, not RNA), crRNA fragment, and tracrRNA fragment and using a long-chain single-stranded DNA as a donor, and found as a surprising result that it is possible to obtain an animal in which the donor DNA has been knocked in with an efficiency of 80% in the verified range (Example 1 to be described later). This was a dramatically higher efficiency than that of the method of Patent Literature 3 described above. Moreover, the method of the present invention made it possible to prepare an individual in which the donor DNA was homozygously knocked in, which is difficult to obtain by the method of Patent Literature 3.

In general, the longer the strand of a donor DNA, the more difficult it is to prepare a knock-in individual. However, it was possible with the method of the present invention to highly efficiently prepare a knock-in individual even in the case of using a single-stranded DNA having a strand longer than in the method of Non Patent Literature 8 (600 bp or more). The method of Non Patent Literature 8 uses a single-stranded DNA of 296 to 514 bp as a donor, and verification under the same condition as in the present invention (single-stranded DNA of 600 bp or more) showed an extremely low knock-in efficiency (Comparative Example 1 to be described later). In light of this, the difference in knock-in efficiency in the method of the present invention as compared with conventional methods is considered to be more noticeable particularly in the case of using a long-chain single-stranded DNA.

In addition, the present inventors have found that it is possible to prepare the long-chain single-stranded donor DNA used in the present invention in a short period of time by a one-tube reaction without limitation on its strand length by a combination of a specific DNA degrading enzyme with asymmetric PCR or unilaterally phosphorylated PCR.

Furthermore, the present inventors have found that the present invention can be widely applied, because of its principle, to systems other than the CRISPR-Cas9 systems as long as they are a genome editing system containing a guide RNA and a protein having nuclease activity, and also that the present invention can be widely applied to cell knock-in for various purposes in addition to knock-in of a donor DNA into a fertilized egg for the purpose of preparing a knock-in animal. These findings have led to the completion of the present invention.

Thus, the present invention provides the following.

[1] A method for preparing a cell in which a desired DNA has been inserted into a target DNA region, comprising the step of introducing

(a) a protein having nuclease activity,
(b) a DNA-targeting RNA containing a nucleotide sequence complementary to a nucleotide sequence of the target DNA region and a nucleotide sequence interacting with the protein (a), and
(c) a single-stranded donor DNA containing the desired DNA into a cell.

[2] A method for preparing a cell in which a desired DNA has been inserted into a target DNA region, comprising the step of introducing

(a) a Cas9 protein,
(b) a combination of a crRNA fragment and a tracrRNA fragment, and
(c) a single-stranded donor DNA containing the desired DNA into a cell.

[3] The method according to [1] or [2], wherein

the single-stranded donor DNA (c) has a nucleotide sequence of 600 bases or more.

[4] The method according to any one of [1] to [3], wherein

the single-stranded donor DNA (c) is prepared by the following method (i) or (ii):

(i) a method including a step of amplifying DNA by PCR using a pair of primers in different amounts and a step of specifically degrading double-stranded DNA contained in an amplified product; and
(ii) a method including a step of amplifying DNA by PCR using a phosphorylated primer as one of the pair of primers and a step of specifically degrading a phosphorylated strand in an amplified product.

[5] The method according to any one of [1] to [4], wherein

the cell is an oocyte.

[6] The method according to [5], wherein

the oocyte is a fertilized egg.

[7] A method for preparing a non-human individual containing a cell in which a desired DNA has been inserted into a target DNA region, the method comprising the steps of:

(a) carrying out the method according to any one of [1] to [6]; and
(b) preparing a non-human individual from a cell obtained in the step (a).

[8] The method according to [7], wherein

the non-human mammal is a rodent.

[9] A kit for use in the method according to any one of [1] to [8], comprising at least one molecule selected from the group consisting of the following (a) to (c):

(a) a protein having nuclease activity;
(b) a DNA-targeting RNA containing a nucleotide sequence complementary to a nucleotide sequence of the target DNA region and a nucleotide sequence interacting with the protein (a); and
(c) a single-stranded donor DNA containing the desired DNA.

### Advantageous Effects of Invention

Even when a single-stranded DNA used as a donor DNA is a long strand, the present invention makes it possible to knock in the single-stranded DNA into cells with a dramatically higher efficiency than that of conventional genome editing techniques. Moreover, it is possible to obtain a cell in which the donor DNA is homozygously knocked in. Additionally, the single-stranded donor DNA used in the present invention can be prepared in a good yield in a short period of time by a one-tube reaction using asymmetric PCR or the like, and no particular limitation is imposed on the strand length of the single-stranded DNA to be prepared. Therefore, the present invention, which uses the combination of the thus prepared single-stranded donor DNA with the cloning-free genome editing system, is a genome editing system that is excellent not only in knock-in efficiency but also in convenience.

### BRIEF DESCRIPTION OF DRAWINGS

FIG. 1 is an electrophoresis photo showing the results of detecting the knock-in efficiency in mice prepared by injecting a long-chain single-stranded donor DNA (floxCol12a1) by an ordinary method (sgRNA+Cas9mRNA). In the knock-in mouse, the band size is shifted to a high molecular weight (hereinafter the same).

FIG. 2 is an electrophoresis photo showing the results of detecting the knock-in efficiency in mice prepared by injecting a long-chain single-stranded donor DNA (Dct-Cre) by an ordinary method (sgRNA+Cas9 mRNA). In the knock-in mouse, the band size is shifted to a high molecular weight.

FIG. 3 is an electrophoresis photo showing the results of detecting the knock-in efficiency in mice prepared by injecting a circular double-stranded donor DNA (floxCol12a1) by a cloning free CRISPR/Cas system (crRNA+tracrRNA+Cas9 protein).

FIG. 4 is an electrophoresis photo showing the results of detecting the knock-in efficiency in mice prepared by injecting a long-chain single-stranded donor DNA (floxCol12a1) by a cloning free CRISPR/Cas system (crRNA+tracrRNA+Cas9 protein).

FIG. 5 is an electrophoresis photo showing the results of carrying out the same experiment as in FIG. 4 by increasing the number of offspring. Note that lane 4 (offspring 3) was unanalyzable.

### DESCRIPTION OF EMBODIMENTS

The present invention provides a method for preparing a cell in which the desired DNA has been inserted into the target DNA region. The method of the present invention comprises the step of introducing the following molecules (a) to (c) into a cell.

(a) Protein Having Nuclease Activity

The “nuclease activity” in the present invention means catalytic activity of cleaving DNA. It is typically an endonuclease activity.

The “protein having nuclease activity” in the present invention binds to the DNA-targeting RNA to be described later and is targeted to the target DNA region, and there is no particular limitation as long as it can exhibit site-specific nuclease activity. Examples of such protein having nuclease activity include Cas9 proteins and Cpf1 proteins. In the present invention, the Cas9 proteins are particularly preferable.

The Cas9 protein in the present invention may be one usable in the CRISPR/Cas9 systems, and binds to a crRNA fragment and a tracrRNA fragment to form a complex and is targeted to the target DNA region to cleave the target double-stranded DNA. Cas9 proteins of various origins are known, and it is possible to use those exemplified in WO 2014/131833 A. It is preferable to use the Cas9 protein derived from Streptococcus pyogenes (SpCas9). The amino acid sequences and nucleotide sequences of the Cas9 proteins are registered in public databases such as GenBank (http://www.ncbi.nlm.nih.gov) (for example, accession No. Q99ZW 2.1 and the like), and it is possible to use these proteins in the present invention.

Preferably, regarding the Cas9 protein in the present invention, it is possible to use one which contains the amino acid sequence represented by SEQ ID NO: 1 or one composed of this amino acid sequence. In addition, it is also possible to use as the Cas9 protein in the present invention a mutant containing an amino acid sequence in which one or more amino acids have been deleted, substituted, added, or inserted to a native amino acid sequence. Here, “more” means 1 to 50, preferably 1 to 30, and more preferably 1 to 10. Moreover, as long as the activity of the original protein is retained, the Cas9 protein in the present invention contains an amino acid sequence having 80% or more, more preferably 90% or more, further preferably 95% or more, and most preferably 99% or more sequence identity with the amino acid sequence represented by SEQ ID NO: 1, or also contains a polypeptide composed of that amino acid sequence. Amino acid sequences can be compared by a known method and, for example, BLAST (Basic Local Alignment Search Tool at the National Center for Biological Information) or the like can be used with the default settings, for example.

Examples of such mutant include a Cas9 protein exhibiting a nickase activity (nCas protein) due to the introduction of a mutation into one of the catalytic sites. Use of an nCas protein makes it possible to reduce off-target effects.

On the other hand, the Cpf1 protein may be one usable in the CRISPR/Cpf1 systems, and binds to a crRNA fragment and is activated to cleave the target double-stranded DNA. In this action, no tracrRNA fragment is required. In addition, there is also a difference in that the Cpf1 protein produces protruding ends while the Cas9 protein produces blunt ends as a result of cleaving the target double-stranded DNA. The Cpf1 proteins are known, and it is possible to use those exemplified in Non Patent Literature 9. It is preferable to use the Cpf1 protein derived from Lachnospiraceae bacterium or Acidaminococcus sp. (LbCpf1 or AsCpf1). Their amino acid sequences are registered in public databases such as GenBank (http://www.ncbi.nlm.nih.gov) (for example, accession Nos.: WP_021736722, WP_035635841, and the like).

For example, regarding the Cpf1 protein in the present invention, it is possible to use one which contains the amino acid sequence represented by SEQ ID NO: 6 or 7 or one composed of this amino acid sequence. In addition, as in the case of the Cas9 protein, it is possible to use a mutant containing an amino acid sequence in which one or more amino acids have been deleted, substituted, added, or inserted to a native amino acid sequence.

In the present invention, the protein having nuclease activity is used in the form of protein. The protein may be produced by a biological method including production by transformed cells or microorganisms obtained by gene recombination technology, or may be chemically produced using a conventional peptide synthesis method. Alternatively, a commercially available product may be used.

(b) DNA-Targeting RNA Containing Nucleotide Sequence Complementary to Nucleotide Sequence of Target DNA Region and Nucleotide Sequence Interacting with Protein (a)

The DNA-targeting RNA of the present invention contains a nucleotide sequence complementary to the nucleotide sequence of the target DNA region and a nucleotide sequence interacting with the protein having nuclease activity.

The term “target DNA region” in the present invention means a region including a site which causes a target gene modification on the genomic DNA of an organism, and is a region composed of usually 17 to 30 bases and preferably 17 to 20 bases. It is preferable that the region be selected from the regions adjacent to the PAM (proto-spacer adjacent motif) sequence on the 3′ side. The site-specific cleavage of the target DNA typically takes place at a position determined by both the complementarity of the formation of base pairs between the DNA-targeting RNA and the target DNA, and the PAM present on the 3′ side thereof.

Although the PAM varies depending on the type and origin of the protein having nuclease activity of the present invention, the PAM is typically “5′-NGG (N is any base)-3′” for Cas9 and is typically “5′-TTN (N is any base)-3′” or “5′-TTTN (N is any base)-3′” for Cpf1. Note that it is also possible to modify PAM recognition by protein modification (for example, by introducing a mutation) (Benjamin, P. et al., Nature 523, 481-485 (2015), Hirano, S. et al., Molecular Cell 61, 886-894 (2016)). This makes it possible to expand the options of target DNA.

Various methods are known as methods for selecting the target DNA region, and it is possible to make a determination by using, for example, the following: CRISPR Design Tool (http://crispr.mit.edu/) (Massachusetts Institute of Technology), E-CRISP (http://www.e-crisp.org/E-CRISP/), Zifit Targeter (http://zifit.partners.org/ZiFiT/) (Zinc Finger Consortium), Cas9 design (http://cas9.cbi.pku.edu.cn/) (Peking University), CRISPRdirect (http://crispr.dbcls.jp/) (University of Tokyo), CRISPR-P (http://cbi.hzau.edu.cn/crispr/) (Huazhong Agricultural University), and Guide RNA Target Design Tool. (https://wwws.blueheronbio.com/external/tools/gRNASrc.jsp) (Blue Heron Biotech).

The DNA-targeting RNA contains a nucleotide sequence which interacts with the protein having nuclease activity (protein binding segment) to form a complex with the protein having nuclease activity (that is, binds by noncovalent interaction). Additionally, the DNA-targeting RNA contains a nucleotide sequence complementary to the nucleotide sequence of the target DNA region (DNA-targeting segment) to give a target specificity to the complex. As described above, the protein having nuclease activity is induced into the target DNA region when it itself binds to the protein binding segment of DNA-targeting RNA, and cleaves the target DNA by its activity.

It is possible to chemically synthesize a DNA-targeting RNA by a method known in the art as a method for synthesizing an oligonucleotide such as the phosphotriethyl method or the phosphodiester method, or by using a usually-used RNA automatic synthesizer.

In the case of the CRISPR/Cas9 systems, DNA-targeting RNA is a combination of crRNA fragment and tracrRNA fragment.

The crRNA fragment at least contains a nucleotide sequence complementary to the nucleotide sequence of the target DNA region and a nucleotide sequence interactable with the tracrRNA fragment in this order from the 5′ side. In the nucleotide sequence interactable with the tracrRNA fragment, the crRNA fragment forms a double-stranded RNA with the tracrRNA fragment, and the formed double-stranded RNA interacts with the Cas9 protein. This guides the Cas9 protein to the target DNA region.

The nucleotide sequence interactable with the tracrRNA fragment means a nucleotide sequence capable of binding (hybridizing) to part of the nucleotide sequence of the tracrRNA fragment. It typically contains at least the nucleotide sequence represented by SEQ ID NO: 2. Consider the case where, in the nucleotide sequence represented by SEQ ID NO: 3, the glycine “G” at the 5′ end is numbered as the first and the subsequent bases are sequentially numbered as the second, the third, the fourth, . . . , and the twenty second. The nucleotide sequence interactable with the tracrRNA fragment preferably contains the nucleotide sequence composed of one or more consecutive bases adjacent to the seventh base, selected from the region of the seventh to twenty second together with the nucleotide sequence composed of the first to sixth bases, or is composed of that nucleotide sequence. Preferably, it contains the nucleotide sequence composed of one or more consecutive bases adjacent to the eleventh base, selected from the region of the eleventh to twenty second together with the nucleotide sequence composed of the first to tenth bases, or is composed of that nucleotide sequence. Indeed, the present inventors have found that the genome editing system of the present invention has cleavage activity when the six bases on the 5′ side are contained, and the genome editing system of the present invention has excellent cleavage activity when the ten bases on the 5′ side are contained in the nucleotide sequence interactable with the tracrRNA fragment.

In addition, the nucleotide sequence interactable with the tracrRNA fragment in the present invention includes an oligonucleotide which is interactable with tracrRNA fragment and which has a nucleotide sequence that binds (hybridizes) under stringent conditions to a nucleotide sequence complementary to the nucleotide sequence described above. The “stringent conditions” mean, for example, hybridization at 65° C. in the presence of 0.7 to 1.0 M NaCl followed by use of an SSC solution at a 0.1- to 2-fold concentration (Saline Sodium Citrate; 150 mM sodium chloride and 15 mM sodium citrate) for washing at 65° C. (also used in the same meaning in the following). Examples of such oligonucleotide can include an oligonucleotide composed of a nucleotide sequence having addition, substitution, deletion, or insertion of a few bases in the above nucleotide sequence (here, “a few bases” mean the number of bases within 3 bases or within 2 bases) and an oligonucleotide composed of a nucleotide sequence having 80% or more, more preferably 90% or more, and most preferably 95% or more identity with the above nucleotide sequence when calculated using BLAST and the like (for example, default, that is, initially set parameters). The present specification refers to such oligonucleotides as “mutant sequences” of the above nucleotide sequence in some cases.

The crRNA fragment contains the nucleotide sequence complementary to the nucleotide sequence of the target DNA region and the nucleotide sequence interactable with the tracrRNA fragment, and can as a whole be composed of preferably 42 bases or less, 39 bases or less, or 36 bases or less or 30 bases or more, 36 bases or more, or 39 bases or more, for example 30 to 42 bases, more specifically 30 bases, 31 bases, 32 bases, 33 bases, 34 bases, 35 bases, 36 bases, 37 bases, 38 bases, 39 bases, 40 bases, 41 bases, or 42 bases.

Note that, in the case of the CRISPR/Cpf1 systems, the DNA-targeting RNA means a crRNA fragment, and the tracrRNA fragment is unnecessary. When the crRNA fragment interacts with the Cpf1 protein, the Cpf1 protein is guided to the target DNA region. In the crRNA fragment, the nucleotide sequence which interacts with the Cpf1 protein (protein binding segment) is disclosed in Non Patent Literature 9.

For the purpose of targeting more than one DNA region and targeting more than one site in the same DNA region, it is possible to use two or more kinds of DNA-targeting RNA. The two or more kinds of DNA-targeting RNA may be simultaneously introduced or continuously introduced into a cell. In the case of using the nCas protein, for example, it is possible to use two or more kinds of DNA-targeting RNA targeting one site for each of the two strands of the target DNA region (a total of two sites).

The tracrRNA fragment in the present invention has a nucleotide sequence capable of binding (hybridizing) to part of the nucleotide sequence of the crRNA fragment on the 5′ side. The interaction of these nucleotide sequences causes the crRNA fragment/tracrRNA fragment to form a double-stranded RNA, and the formed double-stranded RNA interacts with the Cas9 protein.

The tracrRNA fragment in the present invention is not particularly limited as long as it can guide the Cas9 protein together with the crRNA fragment, and tracrRNA derived from Streptococcus pyogenes is preferably used.

Regarding the tracrRNA fragment, the nucleotide sequence necessary for the CRISPR/Cas systems has been made clear to some degree (Jinek et al. Science 337:816, 2012), and it is possible to use these findings in the present invention. The tracrRNA fragment of the present invention contains at least the nucleotide sequence represented by SEQ ID NO: 4. Consider the case where, in the nucleotide sequence represented by SEQ ID NO: 5, the adenine “A” at the 5′ end is numbered as the first and the subsequent bases are sequentially numbered as the second, the third, the fourth, . . . , and the sixty ninth. The tracrRNA fragment preferably contains the nucleotide sequence composed of one or more consecutive bases adjacent to the eleventh and/or the thirty fourth base, selected from the region of the first to tenth and/or the thirty fifth to sixty ninth together with the nucleotide sequence composed of the eleventh to thirty fourth bases, or is composed of that nucleotide sequence.

Therefore, the tracrRNA fragment can as a whole be composed of preferably 69 bases or less, 59 bases or less, or 34 bases or less or 24 bases or more, for example 24 bases, 24 to 34 bases, 24 to 59 bases, or 24 to 69 bases.

In addition, the tracrRNA fragment in the present invention includes an oligonucleotide which is capable of guiding the Cas9 protein together with the crRNA fragment and which has a nucleotide sequence that binds (hybridizes) under stringent conditions to a nucleotide sequence complementary to the nucleotide sequence described above. Examples of such oligonucleotide can include an oligonucleotide composed of a nucleotide sequence having addition, substitution, deletion, or insertion of a few bases in the above nucleotide sequence (here, “a few bases” mean the number of bases within 3 bases or within 2 bases) and an oligonucleotide composed of a nucleotide sequence having 80% or more, more preferably 90% or more, and most preferably 95% or more identity with the above nucleotide sequence when calculated using BLAST and the like (for example, default, that is, initially set parameters). The present specification refers to such oligonucleotides as “mutant sequences” of the above nucleotide sequence in some cases.

(c) Single-Stranded Donor DNA

The single-stranded donor DNA in the present invention is a single-stranded DNA used for inserting (knocking in) the desired DNA into the target DNA region by using homologous recombination (HR) repair occurring at the site cleaved by the protein having nuclease activity.

The process of homologous recombination repair requires homology of the nucleotide sequences of the target DNA and the donor DNA, and the donor DNA is used for template repair of the target DNA (that is, the DNA subjected to double strand break) and genetic information is transferred from the donor DNA to the target DNA. This can result in a change in the nucleotide sequence of the target molecule (for example, insertion, deletion, substitution, and the like).

Hence, the single-stranded donor DNA contains two nucleotide sequences having high identity with the nucleotide sequence in the target DNA region (so-called homology arms) and the desired DNA arranged therebetween (DNA for insertion into the target DNA region).

The homology arms may be of sufficient size for homologous recombination and can vary depending on the strand length of the single-stranded donor DNA, and can be independently selected from the range of, for example, 20 to 300 b. According to the present invention, it is possible to achieve a sufficient knock-in efficiency even with a strand length of, for example, 70 bp or less (for example, 65 bp or less, 60 bp or less, or 55 bp or less).

Moreover, the homology arms need not have 100% identity as long as they have identity with the nucleotide sequence in the target DNA region to a sufficient degree for homologous recombination. For example, they each have 95% or more, preferably 97% or more, more preferably 99% or more, and most preferably 99.9% or more identity when calculated using BLAST and the like (for example, default, that is, initially set parameters).

Furthermore, the size of the desired DNA to be inserted is not particularly limited, and it is possible to use various sizes. The strand length of the single-stranded DNA is, for example, 100 b or more, 200 b or more, 300 b or more, 400 b or more, 500 bp or more, 600 b or more, 700 b or more, 800 b or more, 900 b or more, or 1 kb or more. While Non Patent Literature 8 uses a single-stranded DNA of 296 to 514 bp as a donor, the method of the present invention makes it possible to prepare a knock-in individual with extremely high efficiency even in the case of using a single-stranded DNA having a strand longer than in the method of Non Patent Literature 8 (600 bp or more). Note that, when the method of Non Patent Literature 8 was verified under the condition of the same single-stranded RNA as in the present invention (600 bp or more), the knock-in efficiency was extremely low (Comparative Example 1 to be described later). Therefore, the method of the present invention reveals a significant difference in knock-in efficiency as compared with conventional methods particularly in the case of using a long-chain single-stranded DNA.

If there is a nucleotide sequence to be removed later in the desired DNA, the loxP sequence or the FRT sequence can be added to both ends of that nucleotide sequence, for example. A nucleotide sequence sandwiched between loxP sequences or FRT sequences can be removed by the action of Cre recombinase or FLP recombinase. Additionally, it is possible to incorporate, for example, a selection marker sequence (for example, a fluorescent protein, a drug resistant gene, and the like) into the desired DNA for the purpose of checking the success of the knock-in of the single-stranded donor DNA or the like.

Also, it is possible to operably link a promoter and/or other regulatory sequences to the desired DNA to be inserted. The phrase “operably link” means that the promoter and the desired DNA are linked so as to express the desired DNA (gene) linked downstream of the promoter. The promoter and/or other regulatory sequences are not particularly limited, and appropriately selectable examples thereof include constitutive promoters, tissue specific promoters, timing specific promoters, inducible promoters, CMV promoters, and other regulatory elements (for example, terminator sequences).

Regarding the single-stranded donor DNA of the present invention, it is possible to prepare the single-stranded donor DNA in a short period of time and in a high yield by, for example, a one-tube reaction using the asymmetric PCR method or the unilaterally phosphorylated PCR method.

The asymmetric PCR method is a method for preferentially synthesizing a single strand by making small the amount of one of the pair of primers used in the PCR method. After the asymmetric PCR, the amplified product can be reacted with a double-stranded specific DNA degrading enzyme to degrade the double-stranded DNA, thereby preparing a single-stranded DNA. On the other hand, the unilaterally phosphorylated PCR method uses a phosphorylated primer as one of the pair of primers, which is the primer reverse to the target long-chain single-stranded donor DNA. Then, after the PCR reaction, the amplified product can be reacted with a phosphorylated DNA-specific DNA degrading enzyme to degrade the phosphorylated strand, thereby preparing a single-stranded DNA.

As the primer used in these methods, it is possible to use a primer added with a desired sequence desired as a constituent element of the single-stranded donor DNA. Examples of the desired sequence include, but not limited to, sequences of homology arms and recognition sequences of recombinant enzymes such as the loxP sequence and the FRT sequence.

In addition, preparation is possible by using the method described in Non Patent Literature 8 or the method by Yoshimi et al. (Yoshimi et al., Nat Commun 7:10431 (2016), DOI: 10.1038/ncomms10431).

It is to be understood that the single-stranded DNA of the present invention can be mixed more or less with double-stranded DNA due to the nature of the experiment or for other reasons.

In the case of using two or more kinds of DNA-targeting RNA with different target DNA regions or using two or more kinds of DNA-targeting RNA with different target sites in the same target DNA region in the present invention, it is possible to contain two or more kinds of single-stranded donor DNA having homology arms corresponding to the respective target DNA regions. In this case, it is possible to use DNA of different nucleotide sequences as the desired DNA contained in the two or more kinds of donor DNA.

In the present invention, no particular limitation is imposed on the origin of the cell into which the molecules (a) to (c) are introduced. The cell may be a eukaryotic cell such as an animal cell, a plant cell, an algae cell, and a fungal cell or a prokaryotic cell such as a bacteria or archaea. The eukaryotic cell is preferable and the animal cell is particularly preferable.

Examples of the animal cell include cells of mammals (such as mice, rats, guinea pigs, hamsters, rabbits, humans, monkeys, pigs, cattle, goats, and sheep) as well as cells of fish, birds, reptiles, amphibians, and insects. The cells of mammals are preferably cells of rodents such as mice, rats, guinea pigs, and hamsters and particularly preferably mouse cells.

Moreover, the present invention can target any type of cells, and examples of animal cells include germ cells such as oocytes and sperm; embryonic cells of embryos at various stages (such as 1-cell embryos, 2-cell embryos, 4-cell embryos, 8-cell embryos, 16-cell embryos, and morula embryos); stem cells such as induced pluripotent stem (iPS) cells and embryonic stem (ES) cells; and somatic cells such as fibroblasts, hematopoietic cells, neurons, muscle cells, bone cells, liver cells, and pancreatic cells.

As the oocytes, it is possible to use oocytes before fertilization and after fertilization, preferably oocytes after fertilization, that is, fertilized eggs. Particularly preferably, the fertilized eggs are from pronuclear stage embryos. Oocytes can be thawed and used after freezing.

In the introduction of the molecules (a) to (c) into these cells, it is possible to use known methods for introducing proteins or RNA fragments into cells, and it is possible to preferably use the microinjection method, for example (see, for example, Nagy A, Gertsenstein M, Vintersten K, Behringer R., 2003. Manipulating the Mouse Embryo. Cold Spring Harbour, N.Y.: Cold Spring Harbour Laboratory Press). Other methods such as the electroporation method can also be used.

In the case of microinjection into an oocyte, the microinjection is carried out on the pronucleus or the cytoplasm of the oocyte, or both of them, preferably the pronucleus. In the case of a fertilized egg, the microinjection is carried out on the female pronucleus and/or the male pronucleus, the cytoplasm, or both of them, preferably the male pronucleus.

The conditions for injecting the CRISPR/Cas9 system of the present invention into a cell are as follows, as a representative example.

It is possible to contain in the injection solution the Cas9 protein, the crRNA fragment, and the tracrRNA fragment in amounts selected from the amounts specified in one or more of (i) to (iii) below:

(i) the Cas9 protein is usually 5 to 5000 ng/μL, preferably 5 to 500 ng/μL, more preferably 10 to 50 ng/μL, further preferably 20 to 40 ng/μL, and more further preferably 30 ng/μL;
(ii) relative to 1 ng/μL of the Cas9 protein, the crRNA fragment and the tracrRNA fragment each usually have a concentration exceeding 0.002 pmol/μL, preferably 0.005 pmol/μL or more, more preferably 0.01 pmol/μL or more, and further preferably 0.02 pmol/μL or more and an upper limit concentration of usually 2 pmol/μL or less and preferably 0.2 pmol/μL or less (the amount of the crRNA fragment and the amount of the tracrRNA fragment may be the same or different); and
(iii) the crRNA fragment and the tracrRNA fragment each usually have a concentration exceeding 0.06 pmol/μL, preferably 0.15 pmol/μL or more, more preferably 0.3 pmol/μL or more, and further preferably 0.6 pmol/μL or more and an upper limit concentration of usually 60 pmol/μL or less and preferably 6 pmol/μL or less (the amount of the crRNA fragment and the amount of the tracrRNA fragment may be the same or different).

In one embodiment, the injection solution can contain the Cas9 protein at a concentration of usually 20 to 40 ng/μL and preferably 30 ng/μL, the crRNA fragment at a concentration of usually 0.15 pmol/μL or more, preferably 0.3 pmol/μL or more, and more preferably 0.6 pmol/μL or more, and the tracrRNA fragment at a concentration of usually 0.15 pmol/μL or more, preferably 0.3 pmol/μL or more, and more preferably 0.6 pmol/μL or more.

Those skilled in the art can appropriately set the conditions for using a CRISPR/Cpf1 system based on the conditions for using the CRISPR/Cas9 system described above.

At the time of microinjection, it is preferable that the protein having nuclease activity and the DNA-targeting RNA form a complex. It is possible to form the complex by incubating them in the injection solution usually at 35 to 40° C. and preferably at 37° C. for usually at least about 15 minutes.

The injection volume of the injection solution may be a volume commonly used for microinjection into cells, and can be such a volume that saturates the expansion of the pronucleus of the oocyte in the case of microinjection into the pronucleus, for example.

The injected protein having nuclease activity binds to the DNA-targeting RNA and is guided to the target DNA region on the genomic DNA in the cell, and causes cleavage of the double-stranded DNA in the region.

According to homologous recombination repair, homologous recombination repair takes place in the presence of the donor DNA as a template, and the desired DNA contained in the single-stranded donor DNA can be inserted (gene knock-in) into the target DNA region.

It is possible to introduce the single-stranded donor DNA into a cell together with the protein having nuclease activity and the DNA-targeting RNA, and it can be contained in the injection solution at a concentration of usually 1 to 200 ng/μL and preferably 5 to 10 ng/μL together with other components.

In addition, a combination of two or more kinds of DNA-targeting RNA and two or more kinds of single-stranded donor DNA described above makes it possible to cause several kinds of genetic modifications.

The present invention also provides a method for preparing a non-human individual containing cells in which the desired DNA has been inserted into the target DNA region. This method includes the step of carrying out the above-described method of preparing a cell in which the desired DNA has been inserted into the target DNA region and the step of preparing, from the cell obtained in that step, a non-human individual.

Examples of the non-human individual include non-human animals and plants. Examples of the non-human animals include mammals (such as mice, rats, guinea pigs, hamsters, rabbits, humans, monkeys, pigs, cattle, goats, and sheep) as well as fish, birds, reptiles, amphibians, and insects, preferably rodents such as mice, rats, guinea pigs, and hamsters, and particularly preferably mice. Examples of the plants include cereals, oil crops, feed crops, fruits, and vegetables. Specific examples which can be exemplified include rice, corn, banana, peanut, sunflower, tomato, oilseed rape, tobacco, wheat, barley, potato, soybean, cotton, and carnation.

It is possible to use a known method as a method for preparing anon-human individual from cells. Preparation of a non-human individual from cells of an animal usually employs germ cells or pluripotent stem cells. For example, the molecules (a) to (c) described above are microinjected into an oocyte, and the obtained oocyte is then transplanted into the uterus of a female non-human mammal brought into a pseudopregnant state to then obtain offspring. The transplantation can be carried out for a fertilized egg of 1-cell embryo, 2-cell embryo, 4-cell embryo, 8-cell embryo, 16-cell embryo, or morula embryo. If necessary, it is possible to culture the oocyte subjected to microinjection under appropriate conditions until transplantation. Transplantation and culture for the oocyte can be carried out based on a conventionally known method (Nagy A et al., described above).

In addition, it has long been known that somatic cells of plants possess differentiation totipotency, and methods for regenerating plants from plant cells of various plants have been established. Therefore, for example, it is possible to obtain a plant in which the desired DNA is knocked in by microinjecting the molecules (a) to (c) into plant cells and regenerating plants from the obtained plant cells.

It is possible to confirm the presence or absence of genetic modification and determine the genotype based on a conventionally known method, and examples usable include the PCR method, the sequencing method, and the Southern blotting method.

It is possible to obtain, from the obtained nonhuman individual, progeny or a clone in which the desired DNA has been knocked in.

The method of the present invention makes it possible to genetically modify the target DNA with extremely high efficiency by using a genome editing technique involving a protein having nuclease activity and a DNA-targeting RNA to introduce a single-stranded donor DNA into a cell. The method of the present invention also makes it possible to efficiently produce a non-human mammal having a homozygous genetic modification. Since the present invention exhibits an extremely high knock-in efficiency even in the case of using a long-chain single-stranded DNA of 600 b or more, the present invention makes it possible to knock in genes of various sizes with extremely high efficiency. It is surprising that the knock-in efficiency exhibited was 80% in the verified range when the method of the present invention was used.

The present invention also provides a kit for use in the above-described method of the present invention. The kit of the present invention comprises at least one molecule selected from the group consisting of the protein having nuclease activity, the DNA-targeting RNA, and the single-stranded donor DNA. The kit may include two molecules or may include three molecules.

The kit may further include one or more additional reagents, and examples of the additional reagents include, but not limited to, dilution buffers, reconstitution solutions, wash buffers, and control reagents (for example, DNA-targeting RNA for control).

The elements included in the kit may be accommodated in separate containers or may be contained in the same container. Each of the elements may be accommodated in a container in an amount for single use or may be accommodated in one container in an amount for more than one use (the user can take out the amount necessary for one use). Each element may be accommodated in a container in dry form or may be accommodated in a container in a form dissolved in an appropriate solvent.

### EXAMPLES

Hereinafter, the present invention is described in detail with reference to comparative examples and examples. However, the present invention is not limited to these.

1. Materials and Methods

(1) Design of Long-Chain Single-Stranded Donor DNA

A knock-in mouse designed to lose the target gene only in specific cells having Cre recombinase is called a flox knock-in mouse. Since Cre recombinase has an action of causing loss of a gene region sandwiched between two LoxPs, it is possible to prepare a flox knock-in mouse by sandwiching the target gene with LoxPs. The mouse is considered to be the most difficult in application examples of preparing genetically modified mice by genome editing.

For the purpose of preparing a flox knock-in mouse targeted to the second exon of the mouse Col12a1 gene, the present inventors designed a long-chain single-stranded donor DNA (floxCol12a1 ssDNA, full length of 637 bases) containing the LoxP sequence and the Col12a1 homologous sequence of 55 base pairs at both ends of a gene region containing that exon (SEQ ID NO: 8).

Likewise, for the purpose of preparing a Cre recombinase knock-in mouse targeted to the first exon of the mouse Dct gene, the present inventors designed a long-chain single-stranded donor DNA (full length of 1148 bases) containing Dct homologous sequences of 55 base pairs at both ends of the Cre recombinase gene (SEQ ID NO: 9).

(2) Preparation of Long-Chain Single-Stranded Donor DNA

(i) cDNA Method

The method by Miura et al. (Non Patent Literature 8) was used to prepare a floxCol12a1 long-chain single-stranded donor DNA. Specifically, PCR was used to amplify the gene region containing the second exon of the mouse Col12a1 gene from the mouse genomic DNA using a primer added with the LoxP sequence. All PCR reactions were carried out with PrimeSTAR GXL DNA Polymerase (Takara Bio). This PCR product was used as a template after purification, and a primer added with the Col12a1 homologous sequence of 55 base pairs was further used to prepare by PCR a floxCol12a1 gene cassette. This PCR product was used as a template after purification, and a primer added with a T7 recognition sequence on one side was further used to prepare a T7-floxCol12a1 gene cassette. The nucleotide sequence was confirmed by Sanger sequencing.

Next, this PCR product was used as a template after purification, and mMESSAGE mMACHINE T7 Ultra Transcription Kit (Thermo Fisher Scientific) was used to amplify floxCol12a1 mRNA by the T7 in vitro RNA synthesis method. The floxCol12a1 mRNA was purified with MEGAclear Kit (Thermo Fisher Scientific). Bioanalyzer (Agilient Technologies) and Agilent RNA 6000 Pico Kit (Agilient Technologies) were used for size confirmation by electrophoresis. The concentration was measured with NanoDrop (Thermo Fisher Scientific).

Next, 5 μg of floxCol12a1mRNA was used as a template to synthesize floxCol12a1 cDNA by use of SuperScript III CellsDirect cDNA Synthesis Kit (Thermo Fisher Scientific). The synthesized floxCol12a1 cDNA was separated by agarose gel electrophoresis, and bands of the expected sizes were excised, followed by purification using QIAquick Gel Extraction Kit (Qiagen). The purified floxCol12a1 cDNA was further concentrated by ethanol precipitation. The resultant was the floxCol12a1 long-chain single-stranded donor DNA. The Sanger sequencing method was used to confirm single-stranded DNA and the correctness of the sequence.

(ii) Asymmetric PCR Method

The above-described floxCol12a1 gene cassette PCR product was used as a template to carry out asymmetric PCR with primers designed at both ends (Tm value was 57° C.) Regarding the primers, the primer having a strand same with the DNA amplified as the long-chain single-stranded donor DNA were added at 1 mM finally and the reverse primer was added at 10 to 25 μM finally. PCR was carried out while the primers were annealed at 62° C. for 10 cycles and further at 57° C. for 35 cycles. Agarose gel electrophoresis was carried out to confirm the amplification of the long-chain single-stranded donor DNA. Next, double-stranded specific DNase (PCR decontamination kit, ArcticZymes) was added to the reaction solution after PCR to degrade the double-stranded DNA generated by PCR. MinElute PCR Purification Kit (Qiagen) was used to purify the floxCol12a1 long-chain single-stranded donor DNA. The Sanger sequencing method was used to confirm single-stranded DNA and the correctness of the sequence. The Dct-Cre gene cassette was prepared in the same way.

(iii) Phosphorylated PCR Method

The floxCol12a1 gene cassette PCR product of (i) above was used as a template to carryout ordinary PCR with primers designed at both ends. Note that, regarding the primers, the primer having a strand same with the DNA amplified as the long-chain single-stranded donor DNA was unmodified and the reverse primer had the 5′ end base phosphorylation-modified. The PCR product was confirmed by agarose electrophoresis and purified with QIAquick PCR Purification Kit (Qiagen), and after that, treatment was performed with Lambda Exonuclease (NEB), followed by degradation of only the DNA strands having a phosphorylated 5′ end to obtain a floxCol12a1 long-chain single-stranded donor DNA.

(3) Preparation of Guide RNA

### (i) Cloning Free CRISPR/Cas System Method

The method by Aida et al. (Aida et al., Genome Biol 16:87 (2015), DOI: 10.1186/s13059-015-0653-X) was used for preparation. RNA chemical synthesis was used to prepare crRNAs targeting the 5′ upstream and the 3′ upstream of the second exon of the mouse Col12a1 gene (Fasmac). The cleavage activity of the target DNA was tested by in vitro digestion assay. The crRNAs exhibiting the highest activity for 5′ upstream and the 3′ upstream were used for injection. Likewise, RNA chemical synthesis (Fasmac) was used to prepare a crRNA targeting the first exon of the mouse Dct gene. Likewise, RNA chemical synthesis (Fasmac) was used to prepare a tracrRNA.

Note that the nucleotide sequences of the crRNAs targeting the 5′ upstream and the 3′ upstream of the second exon of the mouse Col12a1 gene are shown at SEQ ID NOs: 10 and 11, and the nucleotide sequence of the crRNA targeting the first exon of the mouse Dct gene is shown at SEQ ID NO: 12. In these crRNAs, 20 bases on the 5′ side serve as a region complementary to the target gene. Additionally, the nucleotide sequence of the tracrRNA is shown at SEQ ID NO: 5.

(ii) Ordinary Method

The method by Aida et al. (described above) was used for preparation. Consider the sgRNA targeting the first exon of the mouse Dct gene and the sgRNAs targeting the 5′ upstream and the 3′ upstream of the second exon of the mouse Col12a1 gene corresponding to the above-described crRNAs. Their T7-added PCR products were used as templates for preparation by the T7 in vitro RNA synthesis method using MEGAshortscript T7 Kit (Thermo Fisher Scientific).

(4) Cas9

(i) Cloning Free CRISPR/Cas System Method

Cas9 proteins (PNA Bio and New England Biolabs) were used.

(ii) Ordinary Method

The method by Aida et al. (described above) was used for preparation. The pX330 (Addgene) plasmid was used to amplify the T7-added Cas9 PCR product, which was used as a template to amplify Cas9 mRNA by the T7 in vitro RNA synthesis method using mMESSAGE mMACHINE T7 Ultra Transcription Kit (Thermo Fisher Scientific).

(5) Injection

The method by Aida et al. (described above) was used for injection. The following injection solution was microinjected into the pronucleus of a frozen fertilized egg of C57BL/6J strain mouse. Cultivation was carried out at 37° C. for a short period of time after injection, followed by transplantation to a pseudopregnant female mouse on the day.

(i) Cloning Free CRISPR/Cas System Method

To a 10 mM Tris-HCl solution, crRNA and tracrRNA (0.61 μM), Cas9 protein (30 ng/μl), and long-chain single-stranded donor DNA (5 to 10 ng/μl) were added.

(ii) Ordinary Method

To a 10 mM Tris-HCl solution, sgRNA (2.5 ng/μl), Cas9 mRNA (5 ng/μl), and long-chain single-stranded donor DNA (5 to 10 ng/μl) were added.

(6) Analysis

The method by Aida et al. (described above) was used for analysis. The knock-in mice were identified by PCR using offspring tail DNA. Accurate knock-in was confirmed by their cloning and Sanger sequence.

2. Results

### [Comparative Example 1] Ordinary Method+Long-Chain Single-Stranded Donor DNA (Method of Non Patent Literature 8)

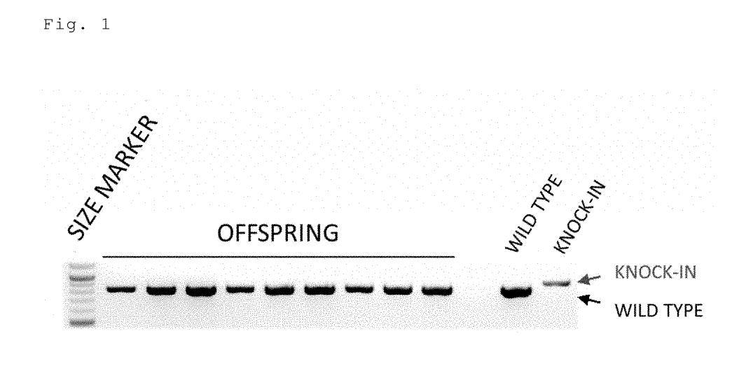

Two sgRNAs targeting the 5′ upstream and the 3′ upstream of the second exon of the mouse Col12a1 gene, a Cas9 mRNA, and a floxCol12a1 long-chain single-stranded donor DNA (637 bases) were injected into a fertilized mouse egg. Of the 26 offspring, no floxCol12a1 knock-in mice were obtained. The knock-in efficiency was 0% (FIG. 1).

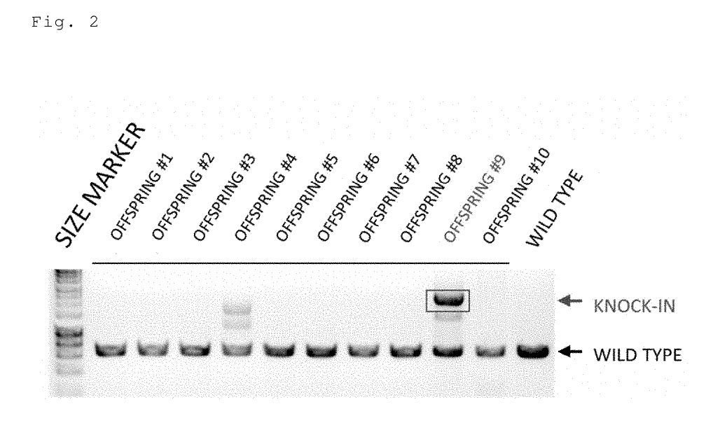

Next, a sgRNA targeting the first exon of the mouse Dct gene, a Cas9 mRNA, a Dct-Cre long-chain single-stranded donor DNA (1148 bases) were injected into a fertilized mouse egg. One of the 10 offspring was a Dct-Cre knock-in mouse. The knock-in efficiency was 10% (FIG. 2).

As described above, it was revealed that a combination of ordinarily used Cas9 mRNA, sgRNA, and long-chain single-stranded donor DNA provides a low knock-in efficiency in the case of using a gene cassette longer than 0.6 kb.

### [Comparative Example 2] Cloning Free CRISPR/Cas System+Circular Double-Stranded Donor DNA

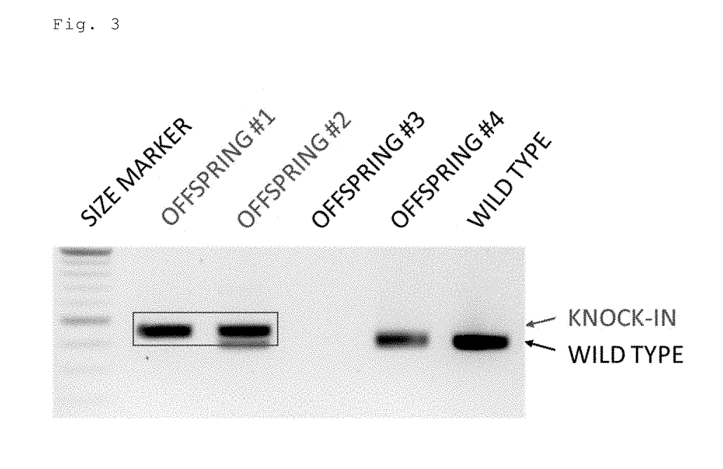

Two crRNAs targeting the 5′ upstream and the 3′ upstream of the second exon of the mouse Col12a1 gene, a tracrRNA, a Cas9 protein, and a floxCol12a1 circular double-stranded donor DNA were injected into a fertilized mouse egg. Three of the 9 offspring were floxCol12a1 knock-in mice. The knock-in efficiency was 33.3% (FIG. 3), but no homo knock-in mice were obtained.

From the above description, use of a cloning free CRISPR/Cas system makes it possible to knock in a gene cassette longer than 0.6 kb. Despite use of a cyclic double-stranded donor DNA of a short homologous region, the knock-in efficiency was somewhat lower than 45.5% shown by Aida et al. (described above), which is generally still highly efficient.

### [Example 1] Cloning Free CRISPR/Cas System+Long-Chain Single-Stranded Donor DNA

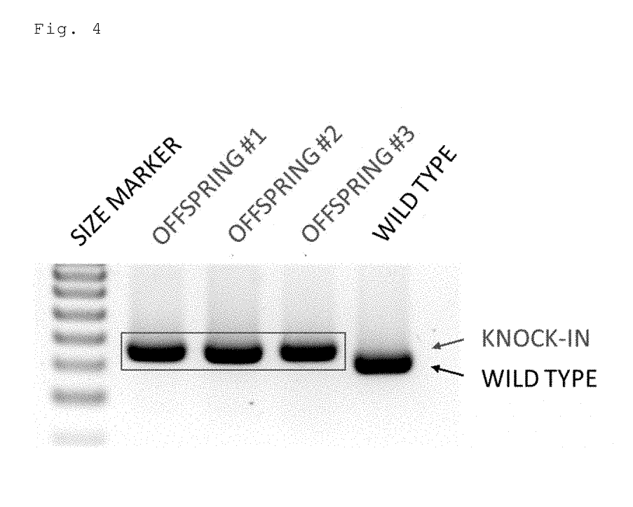

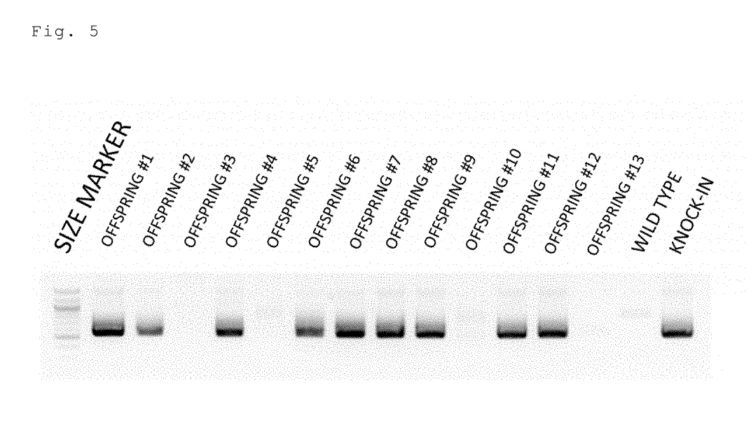

Two crRNAs targeting the 5′ upstream and the 3′ upstream of the second exon of the mouse Col12a1 gene, a tracrRNA, a Cas9 protein, and a floxCol12a1 long-chain single-stranded donor DNA (637 bases) were injected into a fertilized mouse egg. All of the 3 offspring were floxCol12a1 knock-in mice (FIG. 4). One of them was a homo knock-in mouse. Moreover, in the same experiment with an increased number of offspring, 9 of the 12 offspring were floxCol12a1 knock-in mice (FIG. 5; offspring 3 of lane 4 was unanalyzable and thus excluded). The overall knock-in efficiency was 80% (12/15).

Similar experiments were conducted using other genes as targets, and all of these cases confirmed highly efficient knock-in.

As described above, it was revealed that a combination of a cloning free CRISPR/Cas system with a long-chain single-stranded donor DNA makes it possible to extremely highly efficiently knock in a gene cassette longer than 0.6 kb. This greatly exceeds the efficiency of conventional knock-in by cloning free CRISPR/Cas systems and knock-in by a long-chain single-stranded donor DNA. In addition, this combination made it possible to obtain homo knock-in mice for the first time.

### INDUSTRIAL APPLICABILITY

The genome editing system of the present invention makes it possible to highly efficiently prepare a gene knock-in cell. The present invention is expected to be used not only for research purposes such as preparation of experimental animals but also in a wide range of industrial fields, for example medical fields such as regenerative medicine, agricultural fields such as preparation of crops having useful traits, and industrial fields such as production of useful substances utilizing microorganisms.

### SEQUENCE LISTING FREE TEXT

### SEQ ID NOs: 2 to 5 and 10 to 12

<223> Synthetic oligonucleotide

### SEQ ID NOs: 8 and 9

<223> Single-stranded donor DNA

### SEQUENCE LISTING

### IBPF17-519WO-seq-E-fin.txt

1-9. (canceled)

10. A method for preparing a cell in which a desired DNA has been inserted into a target DNA region, comprising the step of introducing
(a) a Cas9 protein,
(b) a combination consisting of two molecules: a crRNA fragment and a tracrRNA fragment, and
(c) a single-stranded donor DNA which contains the desired DNA and has a nucleotide sequence of 600 bases or more
into a cell.

11. The method according to claim 10, wherein
the single-stranded donor DNA (c) is prepared by the following method (i) or (ii):
(i) a method including a step of amplifying DNA by PCR using a pair of primers in different amounts and a step of specifically degrading double-stranded DNA contained in an amplified product; and
(ii) a method including a step of amplifying DNA by PCR using a phosphorylated primer as one of the pair of primers and a step of specifically degrading a phosphorylated strand in an amplified product.

12. The method according to claim 10, wherein the cell is an oocyte.

13. The method according to claim 12, wherein the oocyte is a fertilized egg.

14. A method for preparing a non-human individual containing a cell in which a desired DNA has been inserted into a target DNA region, the method comprising the steps of:
(a) carrying out the method according to claim 10; and
(b) preparing a non-human individual from a cell obtained in the step (a).

15. The method according to claim 14, wherein the non-human mammal is a rodent.

16. A kit for use in the method according to claim 10, comprising at least one molecule selected from the group consisting of the following (a) to (c):
(a) a Cas9 protein,
(b) a combination consisting of two molecules: a crRNA fragment and a tracrRNA fragment, and
(c) a single-stranded donor DNA which contains the desired DNA and has a nucleotide sequence of 600 bases or more.


SEQUENCE LISTING (12 sequences)

SEQ ID NO: 1 - protein, 1368 aa, Streptococcus pyogenes serotype M1 [sequence omitted]

SEQ ID NO: 2 - RNA, 6 nt, Artificial Sequence
  synthetic oligonucleotide
guuuua

SEQ ID NO: 3 - RNA, 22 nt, Artificial Sequence
  synthetic oligonucleotide
guuuuagagcuaugcuguuuug

SEQ ID NO: 4 - RNA, 24 nt, Artificial Sequence
  synthetic oligonucleotide
gcaaguuaaaauaaggcuaguccg

SEQ ID NO: 5 - RNA, 69 nt, Artificial Sequence
  synthetic oligonucleotide
aaacagcauagcaaguuaaaauaaggcuaguccguuaucaacuugaaaaaguggcaccgagucggugcu

SEQ ID NO: 6 - protein, 1307 aa, Acidaminococcus sp. BV3L6
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 7 - protein, 1228 aa, Lachnospiraceae bacterium ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 8 - DNA, 637 bp, Artificial Sequence
  synthetic polynucleotide, single strand donor DNA
ttcctactagtgccctgattaaacctattggaagagcttgacttccatggttccaaagcttataacttcgtatagcatacattatacgaagttatcaatgggtccattatgtgctgggctgggccctgttgttttataggcagatttggctcggttctggtccatgtttaggctctagtctaaccttgttcgtggcctgctcaggctgggttctgatggatcactgcttcctcctgcagatcccttcatgaggctgtgagcttgaggcccaaacatgcagaccaggcttccccgagcgctggccgccctgggcgtggccctactcctgtcttccattgaggcagaaggtaaaatgcttttacttctcctatgacaagtgaaactgcagagttcaagaaaggaaaagactttcatctcctgcttctgttgcttggtctgttttctctgcacttctcaggtgctttctcttcttcagagaaacacctgccttctgaggtgtgtccgtttctgaactttaaaggtccagctcgtttattctgcttccactaataacttcgtatagcatacattatacgaagttatttctgtacagtgctgtgtggtctccttgcgcttgtctctgcagtgtgctgtccac

SEQ ID NO: 9 - DNA, 1148 bp, Artificial Sequence
  synthetic polynucleotide, single strand donor DNA
aaaccaggtgggacgccgggccaggcctcccaattaagaaggcatgggccttgtgatgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattgaaataaaggatgggggcttctgctgggttgtctgggctgcggaattctgctcagagctcggg

SEQ ID NO: 10 - RNA, 42 nt, Artificial Sequence
  synthetic oligonucleotide
ugacuuccaugguuccacaaguuuuagagcuaugcuguuuug

SEQ ID NO: 11 - RNA, 42 nt, Artificial Sequence
  synthetic oligonucleotide
cacagcacuguacagaauagguuuuagagcuaugcuguuuug

SEQ ID NO: 12 - RNA, 42 nt, Artificial Sequence
  synthetic oligonucleotide
gcagaagcccccaucccacaguuuuagagcuaugcuguuuug